(19) United States
(12) Patent Application Publication
(10) Pub. No.: US 2009/0088719 A1
(43) Pub. Date: Apr. 2, 2009

(54) ABSORBENT ARTICLE HAVING INTEGRAL STRETCH PANELS AND METHODS FOR MAKING THE SAME

(75) Inventor: Stacy J. Driskell, Royersford, PA (US)

Correspondence Address: AMSTER, ROTHSTEIN & EBENSTEIN LLP, 90 PARK AVENUE, NEW YORK, NY, 10016, US

(73) Assignee: TYCO HEALTHCARE RETAIL SERVICE AG, Schaffhausen (CH)

(21) Appl. No.: 11/862,774

(22) Filed: Sep. 27, 2007

(51) Int. Cl. A61F 13/15

(52) U.S. Cl. 604/385.101; 604/385.22; 604/385.23

(57) ABSTRACT

Absorbent articles suitable for use as training pants or other hygiene products are provided. The absorbent articles include a chassis having a contiguous front section, a rear section, and a crotch section extending between the front section and the rear section. Edges of the front section connect to edges of the rear section to define a waist opening. The chassis includes a substrate layer having pairs of film sections attached to the front section and the rear section. Each film section is spaced apart from one another and are stretchable in at least a direction along the waist opening. Products according to the invention may provide any of several benefits, including at least one of improved fit, comfort, manufacturability, and visual appearance.

### FIELD OF THE INVENTION

The present invention relates to absorbent articles such as diapers, training pants, and the like. More specifically, the invention relates to absorbent articles having integral side panels that are stretchable in at least one direction.

### BACKGROUND OF THE INVENTION

Absorbent articles such as disposable diapers, training pants, and the like are known for their major function of absorbing and containing body exudates. Such articles are thus intended to prevent the soiling, wetting, or other contamination of clothing or other articles, such as bedding, that come in contact with the wearer. In the case of disposable training pants, they typically include a chassis with a liquid permeable topsheet, a liquid impermeable backsheet, and an absorbent core positioned between the topsheet and the backsheet. Side panels are sometimes attached to the chassis for fitting the training pant about the wearer's waist so the training pant may be pulled up or down like traditional underwear.

While many developments have been made in the art of absorbent articles, there remains a need for further improvements in terms of at least one of improved fit, comfort, manufacturability, and visual appearance.

### SUMMARY OF THE INVENTION

In one aspect, the invention provides an absorbent article having a chassis with a contiguous front section, a contiguous rear section, and a crotch section extending between the front section and the rear section. Edges of the front section are connected to edges of the rear section to define a waist opening. The chassis includes a substrate layer having pairs of film sections attached to the front section and the rear section. Each film section is spaced apart from one another and is stretchable in at least a direction along the waist opening.

In another aspect of the invention, a method of applying a stretchable layer of an absorbent article is provided. The method includes the step of advancing a substrate of the absorbent article in a machine direction. A layer of adhesive is applied to the substrate in a slot coating corresponding to a shape of the stretchable layer. The stretchable layer is then oriented such that it is stretchable in the machine direction. The stretchable layer is attached by means of the adhesive.

### BRIEF DESCRIPTION OF THE DRAWINGS

The invention is best understood from the following detailed description when read in connection with the accompanying drawings, with like elements having the same reference numerals. This emphasizes that according to common practice, the various features of the drawings are not drawn to scale. On the contrary, the dimensions of the various features are arbitrarily expanded or reduced for clarity. Included in the drawings are the following figures:

FIG. 1 is a top plan view of an absorbent article according to an embodiment of the invention.

FIG. 2 is a cross-sectional view of the absorbent article shown in FIG. 1 according to exemplary embodiments of the invention;

FIG. 3 is a top plan view of layers of an absorbent article according to one embodiment of the invention; and

FIG. 4 is a top plan view of layers of an absorbent article according to a second embodiment of the invention.

### DETAILED DESCRIPTION OF THE INVENTION

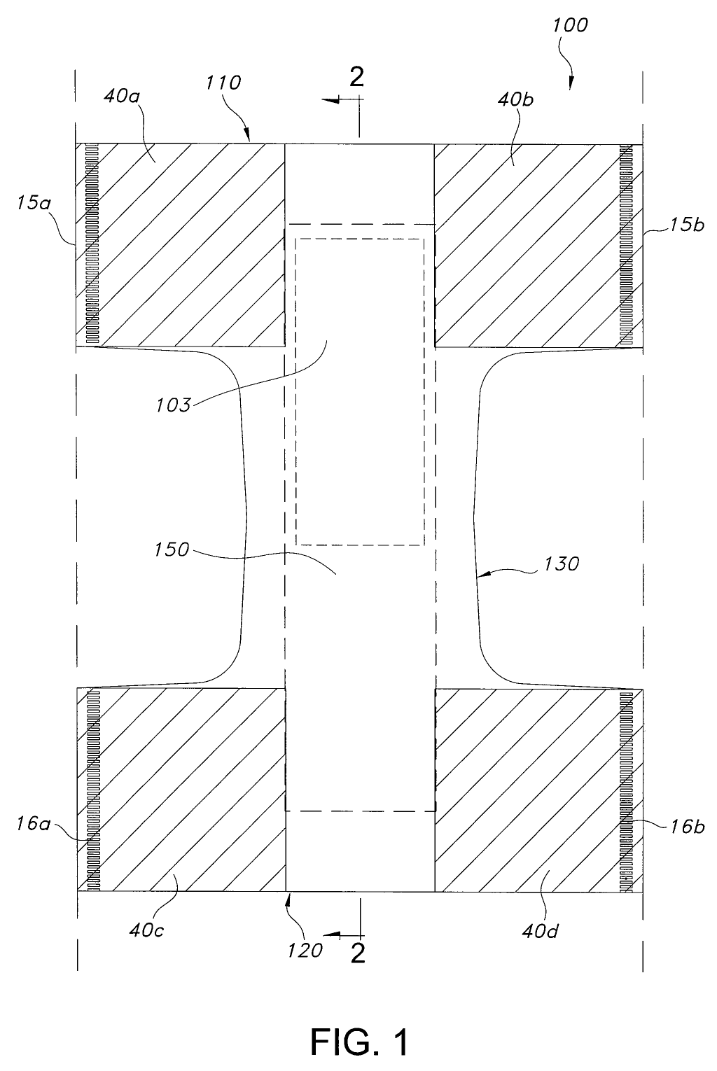

Referring generally to the drawings (FIGS. 1-4), in accordance with an exemplary embodiment, the invention provides an absorbent article 100, 200 having a chassis that includes a contiguous front section 110, 210, a rear section 120, 220, and a crotch section 130, 230 extending between the front section 110, 210 and the rear section 120, 220. Edges 15a, 15b of the front section 110, 210 are connected to edges 16a, 16b of the rear section 120, 220 to define a waist opening. The chassis includes a substrate layer 109 having pairs of film sections 40a-d attached to the front section and the rear section. Each film section 40a-d is spaced apart from one another and is stretchable in at least one direction, such as the machine direction MD, along the waist opening.

In another exemplary embodiment, a method for method of applying a stretchable layer 40 of an absorbent article 100, 200 is provided. The method includes the step of advancing a substrate 109 of the absorbent article 100, 200 in a machine direction MD. A layer of adhesive 1 is applied to the substrate 109 in a slot coating corresponding to a shape of the stretchable layer 40. The stretchable layer 40 is oriented such that it is stretchable in the machine direction MD and the stretchable layer 40 is attached to the substrate 109 by means of the adhesive 1.

Referring now to the drawing in detail, FIG. 1 depicts a plan view of an absorbent article 100 according to one exemplary embodiment of the present invention. Absorbent article 100 includes a chassis having a contiguous front section 110, a rear section 120, and a crotch section 130 extending between the front section 110 and the rear section 120. The chassis has a substantially hour-glass shape such that edges 15a, 15b of the front section 101 to connect to edges 16a, 16b of the rear section 120 to define a waist opening. The edges 15a-b, 16a-b of the front section 110 and rear section 120 may be joined in any suitable manner such as by adhesive bonding, heat sealing, ultrasonic bonding, stitching or the like.

The front section 110 is configured to be positioned against the anterior or front end of a wearer and the rear section 120 is configured to be positioned on the posterior or back end of a wearer. When worn by an individual standing upright, the crotch portion 130 of chassis forms the lowest point on absorbent article 100.

As shown in FIG. 1 and later described, the chassis includes a substrate layer 109 having pairs of stretchable film sections 40a-d integrally attached to the front section 110 and the rear section 120. Each film section 40a-d is spaced apart from one another and is stretchable in at least direction along the waist opening. For example, film sections 40a-d are stretchable in at least the machine direction MD in which absorbent article 100 is manufactured.

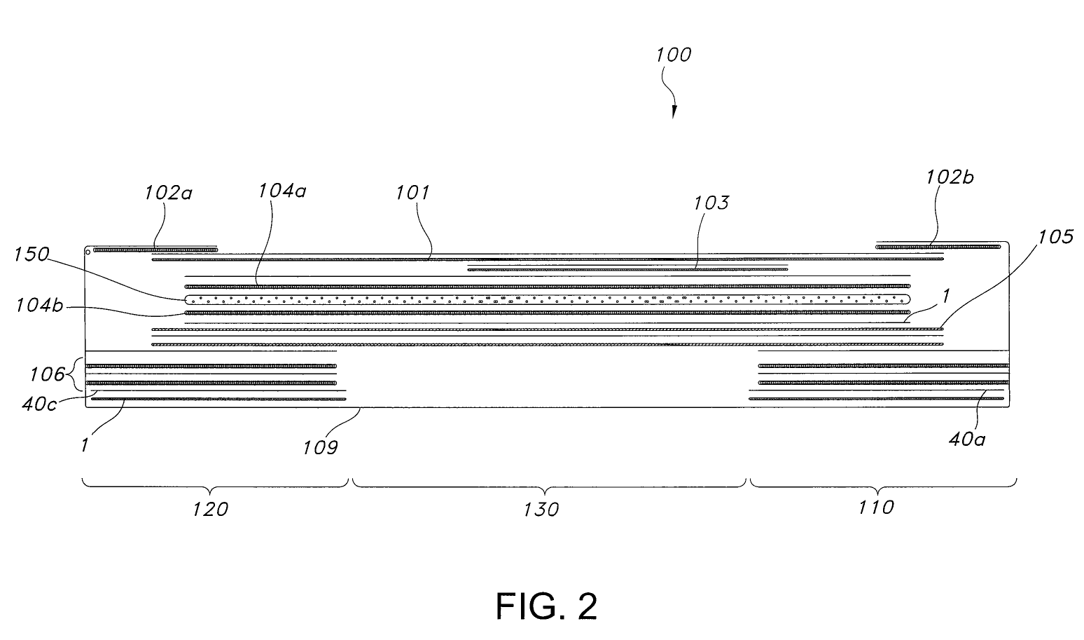

Referring now to FIG. 2, a cross-sectional view of the absorbent article 100 shown in FIG. 1 illustrated. According to an exemplary embodiment, absorbent article 100 includes an absorbent core 150 interposed between a liquid permeable topsheet 101 and a substrate layer 109. In use, the absorbent core 150 is configured to receive liquid through the topsheet 101. When the absorbent article 100 is in place, the liquid permeable topsheet 101 and waist elastic portions 102a-b are arranged to face toward the body of the user (i.e. against the skin of the wearer) and the substrate layer 109 is arranged facing away from the wearer.

In order to enable liquid to quickly and efficiently pass through the topsheet 101 and into the absorbent core 50 for trapping therein, the topsheet 101 is preferably liquid permeable. In particular, the topsheet 101 may be selected from a variety of textile-like films and fabrics. Suitable fabrics include non-woven materials that are pervious to liquid, soft and pliable. Preferred non-woven materials include spun-bonded polypropylene, spunbonded polyethylene, and thermally bonded webs of staple fibers, preferably polypropylene shape or sheath/core bi-component fibers having a core of polyester or polypropylene and a sheath of polyethylene.

As seen with respect to the embodiment of the absorbent article 100 shown in FIG. 1, the absorbent core 150 which is interposed between the topsheet 101 and substrate layer 109 may be of any shape, but is typically a rectangular member that is centered in the chassis and extends from a portion of the front section 120 to a portion of the rear section 120. The absorbent core 150 may be made of any suitable absorbent material, as well as combinations of different types of absorbent materials. For example, the absorbent core 138 may be formed of a mixture of pulp fluff and superabsorbent polymer (SAP) wrapped in a liquid permeable tissue wrap 104a-b. Examples of SAP include polyacrylamides, polyvinyl alcohol, polyacrylates, various grafted starches, and the like. The super absorbent materials can be in various geometric forms, such as various shaped particles, fibers, foams, and layers. The amount of each absorbent material and SAP/fluff ratio depends on factors including the size of the article 100.

According to an exemplary embodiment, the chassis includes an acquisition layer 103 interposed between the topsheet 101 and absorbent core 150 to rapidly take up fluid through the topsheet 101 and then release it into the absorbent core 150. Acquisition layer 103 remains substantially dry even after liquid has penetrated the acquisition layer 103. Thus, acquisition layer 103 provides an insulating layer between the absorbent core 150 and topsheet 101 to prevent contact between a wet absorbent core 150 and the wearer's skin.

The acquisition layer 103 is generally composed of resilient fibrous webs that provide a small absorbent volume to rapidly take up liquids and then direct it to the absorbent core 106. This allows adequate time for super absorbent materials in the core 150 to absorb the liquid. The thickness of the acquisition layer 103 is preferrably thin to provide the small absorbent volume.

A liquid impermeable barrier layer 105 is interposed between the absorbent core 150 and substrate layer 109. Barrier layer 105 is arranged facing away from the wearer (i.e. away from the skin of the wearer) and is preferably formed from a hydrophobic polymeric film to prevent liquid from seeping through absorbent core 150 and wetting a wearer's clothing. Materials for forming the barrier layer 105 may include polyethylene films, polypropylene films, co-extruded films (polyethylene and ethylene vinyl acetate), co-polymer films (polyethylene/polypropylene), and polylaminates (polypropylene nonwoven and polyethylene film). In an exemplary embodiment, barrier layer 105 may be joined to tissue 104b of absorbent core 150 in any suitable manner such as hot melt adhesive 1.

According to the present invention as shown in FIG. 2, stretch panels are integrally formed on absorbent article 100 by attaching stretchable, non-porous film sections 40a, 40c to substrate layer 109. Each film section 40a, 40c is attached directly to substrate layer 109 by a layer of adhesive 1. Adhesive 1 may be applied to substrate layer 109 in various patterns. For example, adhesive 1 may be applied in a spiral, comb, or omega pattern. Preferably, adhesive 1 is applied in a slot coating such that the dimensions of the adhesive 1 correspond to the shape of the film section 40a, 40c. Thus, when film section 40a, 40c is adhered to substrate layer 109, the stretch panels form a smooth appearance. In another exemplary embodiment, film section 40a, 40c may be joined to substrate layer 109 in any suitable manner such as by heat sealing, ultrasonic bonding, stitching or the like.

In an exemplary embodiment, film section 40a, 40c are stretched prior to adhering the film section 40a, 40c on substrate layer 109. Thus, after film sections 40a, 40c have been attached, stretch panels “retract” due to tension from the film sections 40a, 40c. Each film section 40a, 40c is spaced apart from one another such that when edges 15a-b, 16a-b (FIG. 1) of the front section 110 and rear section 120 are connected, stretch panels are located near the hips of wearer. In this embodiment, middle portions of the front section 110 and rear section 120 are substantially non-stretchable, thereby providing a substantially smooth surface in which indicia may be printed or attached. In an exemplary embodiment, cloth-like carrier layers 106 may be attached above film sections 40a, 40c to provide additional padding and form the stretch panel laminate. A portion of substrate layer 109 may be folded over and adhered to carrier layer 106 and/or waist elastic portion 102a to complete the finished absorbent article 100 product.

According to an exemplary embodiment, film sections 40a, 40c may be made of any thermoplastic elastomer (TPE). Such resins/copolymers may include, for example, styrene-ethylene/butylene-styrene (SEBS) or styrene-ethylene/propylene-styrene (SEPS) that may be used to create films that are extrudable at higher temperatures then become elastic at an ambient temperature. The stretchable film sections may be breathable or non-breathable depending on the desired properties of the absorbent article 100. For example, a stretchable film made of a “breathable” microporous polyethylene to provided added comfort and passage of air through the stretch panel laminate.

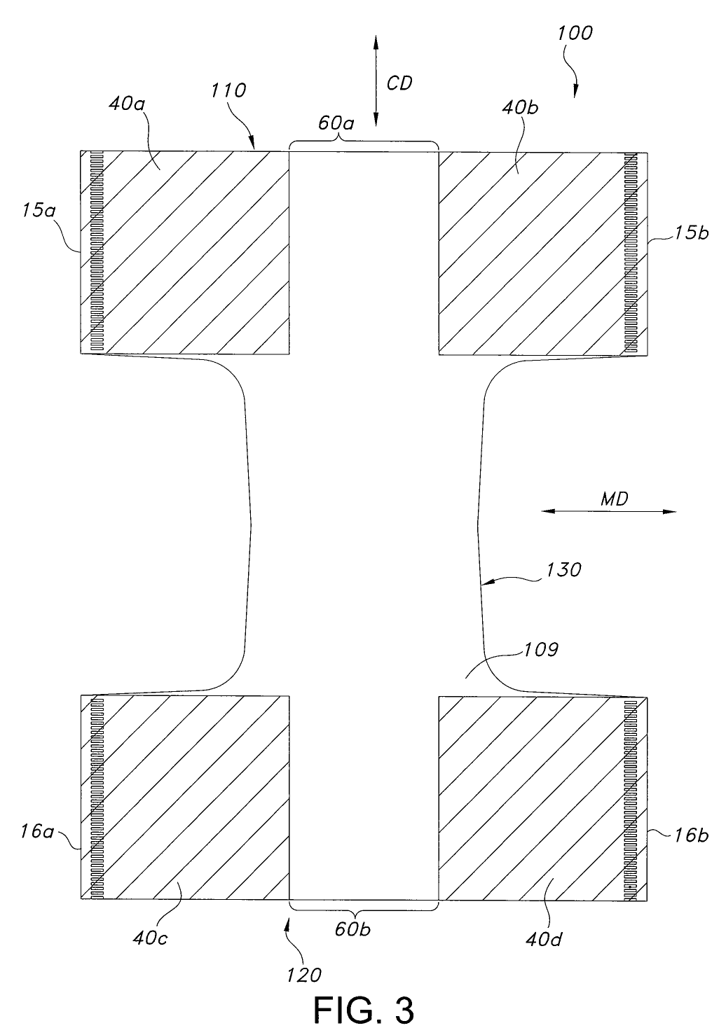

Referring now to FIG. 3, the substrate layer 109 of absorbent article 100 shown in FIGS. 1 and 2 is illustrated. As described above, film sections 40a-d are integrally attached to substrate layer 9 by adhesive, thereby forming the stretch panel of absorbent article 100. In an exemplary embodiment, film sections 40a-d are stretched in the machine direction MD (i.e., direction of manufacture) prior to attachment on substrate layer 109. For example, film sections 40a-d may be stretched by a series of rollers and cut prior to being adhered to substrate layer 109 with spaces between film sections 40a-d. As described above, portion 60a, 60b in which film sections 40a-d are not attached may be substantially non-stretchable such that indicia may be printed or attached to substrate layer 109.

In an exemplary embodiment, film sections 40a-d may be stretched in a direction perpendicular to the machine direction MD such that film sections 40a-d are stretchable in at least two directions. In this embodiment, film sections 40a-d may be stretched in both the cross-direction CD and machine direction MD, to provide and absorbent article 100 with integral panels that are stretchable in at least two directions. According to another embodiment, film sections 40a-d may be oriented with respect to substrate layer 109 such that the film sections 40a-d are stretchable in the machine direction MD. For example, rolls of substrate layer 109 and/or stretchable film layer 40a-d may be oriented parallel to each other using a machine direction orienter such that as substrate layer 109 is running in the machine direction MD, the stretchable layer may be stretched and attached to the substrate layer 109 in the same direction.

In an exemplary embodiment, the substrate layer 109 may be a non-elastic material may be formed from a single layer of non-woven material, and may exhibit substantially no elastic/stretch properties (i.e. inelastic). The substrate layer 109 is optionally formed from a laminated sheet of a non-woven material and film (with the non-woven side positioned as the outermost layer). One particularly suitable material is a spunbond-meltblow-spunbond (SMS) web. The spunbond layer is optionally made of polypropylene fibers. Such composites provide the dual advantages of liquid barrier properties of film along with a soft, outer fabric texture.

The substrate layer 109 can also be made of other suitable cloth-like materials, e.g., spun-bond or thermal-bond non-woven web made of either polypropylene, polyethylene, polyester, bi-component fibers (polyethylene/polypropylene or polyethylene/polyester), or any combinations of these fibers. Various multiple layer configurations or fiber denier variations may be used. Another example includes hydro-entangled non-woven webs, which may contain some cotton and/or rayon fibers blended in with thermal-plastic fibers. Cellulose fibers can also be blended in at small percentages to reduce cost. Still, other materials for forming the substrate layer 109 may include polypropylene films, co-extruded films (polyethylene and ethylene vinyl acetate), co-polymer films (polyethylene/polypropylene), and polylaminates (polypropylene nonwoven and polyethylene film). In yet another embodiment, the substrate layer 109 is optionally selected from a variety of textile-like films and fabrics.

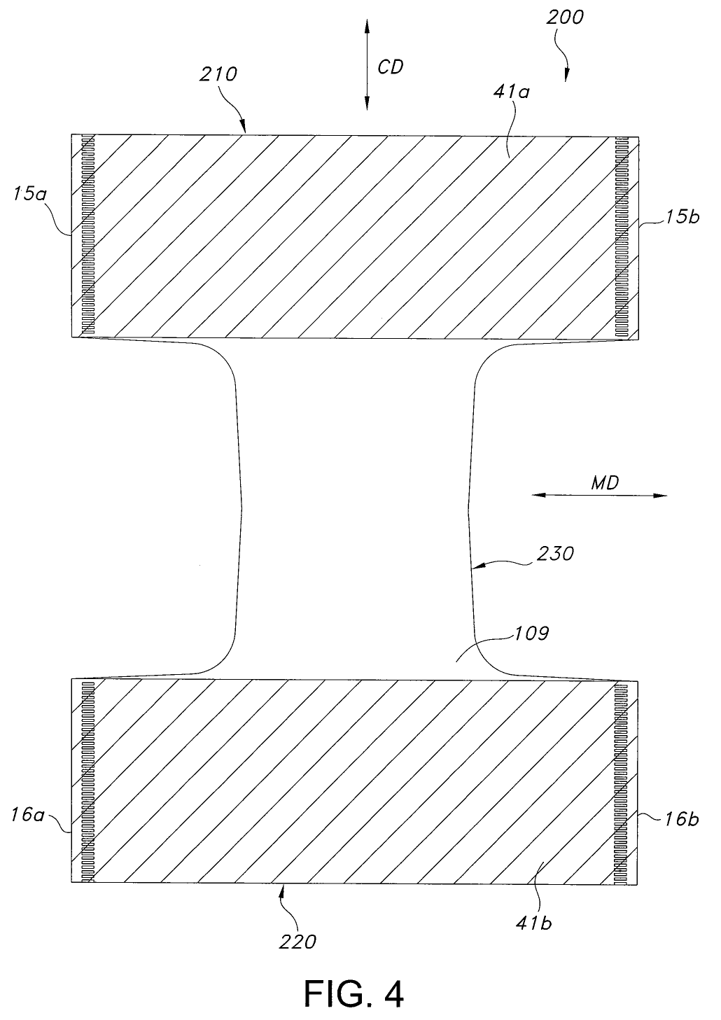

Referring now to FIG. 4, according to an exemplary embodiment, absorbent article 200, includes a chassis in which stretchable layer 41a, 41b is attached to substrate layer 109 across the entire front section 210 and rear section 220. Thus, the entire waist portion of absorbent article 200 is stretchable in at least one direction. For example, the entire waist portion of absorbent article 200 is stretchable in the machine direction MD, cross-direction CD, or both the machine direction MD and cross-direction CD.

Although the present invention has been particularly described in conjunction with specific embodiments, it is evident that many alternatives, modifications, and variations will be apparent to those skilled in the art. It is therefore contemplated that the appended claims will embrace any such alternatives, modifications, and variations as falling within the true scope and spirit of the present invention.

What is claimed:

1. An absorbent article comprising:
a chassis having a contiguous front section, a contiguous rear section, and a crotch section extending between the front section and the rear section, edges of the front section being connected to edges of the rear section to define a waist opening,
wherein the chassis comprises a substrate layer having a pair of film sections attached to each of the front section and the rear section, the film sections of each of the pairs being spaced apart from one another and being stretchable in at least a direction along the waist opening.

2. The device of claim 1, wherein the chassis further comprises an absorbent core interposed between the substrate layer and a liquid permeable topsheet, the absorbent core configured to receive liquid through the topsheet.

3. The device of claim 2, wherein the chassis further comprises an acquisition layer interposed between the topsheet and the absorbent core, the acquisition layer configured to receive liquid through the topsheet and direct liquid to the absorbent core.

4. The device of claim 2, wherein the chassis further comprises a liquid impermeable barrier layer interposed between the absorbent core and substrate layer.

5. The device of claim 1, wherein the substrate layer has a substantially hour-glass configuration.

6. The device of claim 1, wherein the substrate layer is made of a non-woven material.

7. The device of claim 1, wherein each film section is nonporous.

8. The device of claim 1, wherein each film section is stretchable in a direction transverse to the waist opening.

9. The device of claim 1, wherein each film section is stretchable in a machine direction and in a cross-direction transverse to the machine direction.

10. A method of applying a stretchable layer of an absorbent article comprising the steps of:
advancing a substrate of the absorbent article in a machine direction;
applying a layer of adhesive to the substrate in a slot coating corresponding to a shape of the stretchable layer;
orienting the stretchable layer such that it is stretchable in the machine direction; and
attaching the stretchable layer to the substrate by means of the adhesive.

11. The method of claim 10, further comprising stretching the stretchable layer in a stretched conditioned such that the stretchable layer corresponds to a shape of the layer of adhesive.

12. The method of claim 11, wherein the step of stretching comprises stretching the stretchable layer in the machine direction and in a cross direction traverse to the machine direction.

13. A method of forming an absorbent article comprising the steps of:
providing a substrate layer of a chassis having a contiguous front section, a contiguous rear section, and a crotch section extending between the front section and the rear section;
attaching a pair of film sections to each of the front section and the rear section such that the film sections of each of the pairs is spaced apart from one another and stretchable in at least a direction along the waist opening; and
connecting edges of the front section to edges of the rear section to define a waist opening.

14. The method of claim 13, further comprising the step of advancing a substrate of the absorbent article in a machine direction.

15. The method of claim 13, wherein the attaching step comprises at least one step consisting of adhesive bonding, heating sealing, or ultrasonic bonding the pair of film sections to each of the front section and the rear section.

16. The method of claim 13, further comprising the step of applying a layer of adhesive to the substrate in a slot coating corresponding to a shape of the stretchable layer.

17. The method of claim 13, further comprising the step of orienting the stretchable layer such that it is stretchable in the machine direction.
